SEQUENCE LISTING (1 sequence)

SEQ ID NO: 1 - DNA, 45 bp, Artificial Sequence
  chemically synthesized; probe
ttgttacaagggactttccgctggggactttccaggaggcgtggg

(19) United States
(12) Patent Application Publication
(10) Pub. No.: US 2010/0190733 A1
(43) Pub. Date: Jul. 29, 2010

(54) Methods of Treating Muscular Dystrophies

(75) Inventors: Francesco Squadrito, Messina (IT); Alessandra Bitto, Messina (IT); Giuseppe Vita, Messina (IT); Sonia Messina, Messina (IT); Bruce P. Burnett, Hollywood, FL (US)

Correspondence Address: PATENT CORRESPONDENCE;ARNALL GOLDEN GREGORY LLP, 171 17TH STREET NW, SUITE 2100, ATLANTA, GA, 30363, US

(73) Assignee: Primus Pharmaceuticals, Inc., Scottsdale, AZ (US)

(21) Appl. No.: 12/693,711

(22) Filed: Jan. 26, 2010

Related U.S. Application Data
(60) Provisional application No. 61/147,460, filed on Jan. 26, 2009.

(51) Int. Cl. A61K 31/352; A61P 21/00; A61K 31/7048; A61K 31/353

(52) U.S. Cl. 514/27; 514/456

(57) ABSTRACT

The invention relates to methods of treating Duchenne muscular dystrophy with flavonoids. The methods may include (a) providing a pharmaceutical composition comprising a therapeutically effective amount of flavonoid, and (b) administering the composition to a human patient, wherein the flavonoid comprises an isoflav-4-one with at least one of the carbons located at positions 8, 7, 6, 5, 2, 2′, 3′, 4′, 5′, or 6′ modified by an alcohol group. Alternatively, the flavonoid may comprise (1) a flavan-3-ol with at least one of the carbons located at positions 8, 7, 6, 5, 4, 2′, 3′, 4′, 5′, or 6′ modified by an alcohol group, or (2) a Free-B-Ring flavone with at least one of the carbons located at positions 8, 7, 6, 5 or 3 modified by an alcohol group and with at least one of the carbons located at positions 8, 7, 6, 5 or 3 modified by a glycoside group.

### RELATED APPLICATIONS

This application claims priority to U.S. Provisional Application No. 61/147,460, filed Jan. 26, 2009. The contents of said application are incorporated by reference as if fully set forth herein.

### FIELD OF THE INVENTION

The present invention relates to methods of treating Duchenne muscular dystrophy and other muscular dystrophies with flavonoids.

### BACKGROUND OF THE INVENTION

Duchenne muscular dystrophy (DMD) is a severe, X-chromosome linked hereditary disease characterized by the rapid progression of muscle degeneration, leading to paralysis, loss in ambulation, and death, usually in early adulthood. The disease affects 1 in 3500 males, making it the most prevalent of muscular dystrophies (See Ref. 1 below).

DMD is caused by the absence of the protein dystrophin, an essential structural component of the dystrophin-glycoprotein complex which maintains the integrity of muscle fibers. Although the dystrophin genetic defect is known to cause DMD, the specific mechanisms responsible for the pathological hallmarks of the DMD, such as necrosis, exhaustible regeneration, and secondary fibrosis, are not completely understood.

It is believed that loss of muscle fiber integrity caused by the absence of dystrophin progresses to myofiber necrosis, phagocytosis, and infiltration of the muscle by inflammatory cells. Regeneration from satellite cells is not sufficient to replace necrotic muscle fiber, so as the disease progresses, the loss of muscle fibers and subsequent fibrosis leads to impaired muscle function (See Ref. 2 below).

The mdx mouse, a genetically homologous DMD model, is frequently used to study the disease pathogenesis. In the mdx mouse, the lack of dystrophin has been shown to severely compromise the mechanical integrity of myofibers and to increase vulnerability to mechanical stress, hypo-osmotic shock, and contraction-induced damages. As compared to the human disease, the murine model shows slower disease progression, similar repetitive degeneration and regeneration cycles occurring between 2 and 12 weeks of age, muscle weakness at a later stage, but no proliferation of connective tissue in limb muscles. (See Ref. 3 and 4 below).

Several lines of evidence have suggested the involvement of oxidative stress in the dystrophic process. Dystrophic muscle cells are known to be more susceptible to reactive oxygen intermediates (ROIs) (See Ref. 5 and 6 below). Moreover, it is known that free radical injury contributes to membrane integrity loss in muscular dystrophies, and that markers of oxidative stress have been detected in muscles of DMD patients and mdx mice (See Ref. 5 and 6 below). Furthermore, increased biological by-products of oxidative stress, reduced cellular antioxidants (glutathione and vitamin E), and altered concentrations of antioxidants enzymes have been found in DMD (See Ref. 5, 6 and 7 below).

Traditionally, it has been believed that oxidants exert their effects via a direct toxic action on target cells. However, recent studies have suggested their contributory role in gene induction. For example, it has been suggested that NF-κB, a pleiotropic transcription factor activated by low levels of ROIs and inhibited by antioxidants, has a role in the muscle-wasting process. NF-κB is a major transcription factor that regulates the expression of a plethora of genes involved in inflammatory, immune, and acute stress responses (See Ref. 8). In unstimulated cells, NF-κB is inactive via interaction with its inhibitor protein (I-κB). NF-κB activation is regulated by an I-κB kinase (IKK) complex composed of catalytic subunits, IKKα and IKKβ, and a regulatory subunit IKKγ/NEMO (See Ref. 9). Classical stimulatory signals such as proinflammatory cytokines result in IKKβ-mediated site-specific phosphorylation and subsequent degradation of I-κB. Loss of I-κB allows nuclear NF-κB entry and subsequent transcription of diverse set of genes encoding growth factors, cytokines, chemokines, antiapoptotic proteins and cell adhesion molecules (See Ref. 10). Specifically, NF-κB, after proteasomal degradation of the inhibitory protein I-kappa-B (IκB-α), translocates to the nucleus and binds target DNA elements in the promoter of different genes expressing cytokines, chemokines, cell adhesion molecules, immunoreceptors, and inflammatory enzymes such as nitric oxide synthase, matrix metalloproteinases, and phospholipase A2 (See Ref. 11-14).

NF-κB activity has been demonstrated to be increased in muscles of DMD patients and mdx mice (See Ref. 15-18). Increased immunoreactivity for NF-κB has been reported in the cytoplasm of all regenerating fibers and in 20-40% of necrotic fibers in DMD as well as in inflammatory myopathies (See Ref. 15). It also is known that NF-κB is activated in response to several inflammatory molecules such as interleukin-1β (IL-1β), tumor necrosis factor-α (TNF-α), and metalloproteinases that cause muscle loss and whose circulating levels have been found elevated in DMD and other types of muscular dystrophies (See Ref. 11, 16 and 19-20). Furthermore, NF-κB regulates myogenesis, and systemic administration of the NF-κB inhibitor curcumin stimulates muscle regeneration after traumatic injury, suggesting a beneficial effect of NF-κB blockade on muscle repair (See Ref. 21).

It has been reported that increased NF-κB activity occurs in DMD muscle with an augmented immunoreactivity in the cytoplasm of all regenerating fibers and in 20 to 40% of necrotic fibers (See Ref. 22). This increase also was confirmed in mdx mice muscle (See Ref. 23). Several lines of evidence have demonstrated that NF-κB is strongly activated by oxidative stress, and its activation can lead to augmented expression of inflammatory molecules such as IL-113, COX-2 and TNF-α which are all involved in the dystrophic process and the promotion of muscle wasting (See Ref. 24-30). The activation of NF-κB is regulated by cellular kinases, including mitogen-activated protein kinases (MAPKs), a family of serine/threonine protein kinases responsible for the phosphorylation of variety of effector proteins (See Ref. 31-32). Members of the MAPKs family, such as p38 and c-Jun NH2-terminal kinase 1 (JNK1), have been postulated to play an important role in regulating TNF-α gene expression as well as contributing to the dystrophic process (See Ref. 33-35).

In previous studies, the inhibition of NF-κB with compounds such as pyrrolidine dithiocarbamate (PDTC) and (±)-5-emisuccinoyl-2-[2-(acetylthio)ethyl]-2,3-dihydro-4,6,7-trimethylbenzofuran (IRFI-042), both potent antioxidants, has been shown to have beneficial effects on functional, biochemical and morphological parameters of mdx mice muscle fibers (See Ref. 36-37). It also has been shown that green tea extract and its component epigallocatechin gallate have a beneficial effect on mdx mice (See Ref. 38 and 39). However, specific methods of treating DMD have yet to be perfected, in part due to the thousands upon thousands of different molecules that may or may not be useful in treating DMD.

### SUMMARY OF THE INVENTION

The present invention relates to methods of treating muscular dystrophies, especially Duchenne muscular dystrophy, with flavonoids.

Flavonoids are diverse of compounds that are found in abundance in a large variety plants and herbs. Over 9000 flavonoids have been either synthesized or isolated from natural sources (See Ref. 40). To date, antioxidants such as flavonoids have been used to treat an extremely wide variety of health problems including fatigue, heart disease, brain injury, and neurological diseases.

Those familiar with the art recognize that flavonoids products are generally known for their antioxidant activity. However, the precise biological properties of specific types of flavonoids are poorly understood. Unfortunately, it is still very difficult to predict which of the large variety of flavonoid molecules may be useful for treating each of the large number of potential health conditions that could be ameliorated with antioxidants.

This invention therefore is based on the unexpected discovery that specific types of flavonoids may be used effectively to treat muscular dystrophies such as Duchenne muscular dystrophy. It certainly has never been expected that these particular types of flavonoids would be effective in treating this specific type of medical condition.

In one embodiment, the method of treating muscular dystrophies, especially Duchenne muscular dystrophy, includes (a) providing a pharmaceutical composition comprising a therapeutically effective amount of flavonoid, and (b) administering the composition to a human patient, wherein the flavonoid comprises an isoflav-4-one nucleus, and wherein at least one of the carbons of the nucleus located at positions 8, 7, 6, 5, 2, 2′, 3′, 4′, 5′, or 6′ is modified by an alcohol group.

In another embodiment, the method of treating muscular dystrophies, especially Duchenne muscular dystrophy, includes (a) providing a pharmaceutical composition comprising a therapeutically effective amount of flavonoid, and (b) administering the composition to a human patient, wherein the flavonoid comprises a flavan-3-ol nucleus, and wherein at least one of the carbons of the nucleus located at positions 8, 7, 6, 5, 4, 2′, 3′, 4′, 5′, or 6′ is modified by an alcohol group.

In yet another embodiment, the method of treating muscular dystrophies, especially Duchenne muscular dystrophy, includes (a) providing a pharmaceutical composition comprising a therapeutically effective amount of flavonoid, and (b) administering the composition to a human patient, wherein the flavonoid comprises an isoflav-4-one nucleus, and wherein at least one of the carbons of the nucleus located at positions 8, 7, 6, 5, 2, 2′, 3′, 4′, 5′, or 6′ is modified by an alcohol group

### BRIEF DESCRIPTION OF THE DRAWINGS

FIG. 1 is set of graphs showing the effects of flavocoxid, methylprednisolone and vehicle treatment on body weight (A), forelimb strength (B), forelimb strength normalized to weight (C) and fatigue (D) in WT and mdx mice. §=p<0.01 and *=p<0.05 vs vehicle treated mdx mice.

FIG. 2 is a graph showing the effects of flavocoxid, methylprednisolone and vehicle treatment on serum CK levels in WT and mdx mice. §=p<0.01 vs vehicle treated mdx mice.

FIG. 3 is a set of graphs showing the surface area of biceps muscles occupied by normal area (A), necrotic area (B), regenerating area (C) and centrally nucleated fibers area (D) in response to flavocoxid, methylprednisolone and vehicle treatment in WT and mdx mice. §=p<0.01 and *=p<0.05 vs vehicle treated mdx mice.

FIG. 4 is a set of graphs showing numbers of myogenin-positive SC (A), myogenin-positive nuclei (C,) and dMHC-positive fibers (E) per mm² in biceps muscles of WT and mdx mice in response to flavocoxid, methylprednisolone and vehicle treatment. *=p<0.05 vs vehicle treated mdx mice.

FIG. 5 is a graph showing quantitative data from an electrophoretic mobility shift assay of muscular NF-κB binding activity of WT and mdx mice in response to flavocoxid, methylprednisolone and vehicle treatment. §=p<0.01 and *=p<0.05 vs vehicle treated mdx mice. The figure also includes a representative autoradiogram.

FIG. 6 is a set of graphs showing levels of muscular H₂O₂ (A) and total peroxidase activity (B) in response to flavocoxid, methylprednisolone and vehicle treatment in WT and mdx mice. *=p<0.05 vs vehicle treated mdx mice.

FIG. 7 is a set of graphs showing quantitative data from a western blot for COX-2 (A), 5-LOX (B), phospho-p38 (C), phospho-JNK1 (D) and TNF-α (E) of WT and mdx mice in response to flavocoxid, methylprednisolone and vehicle treatment. §=p<0.01 vs vehicle treated mdx mice. The figure also includes representative autoradiograms.

FIG. 8 is a graph showing the forelimb strength of mdx mice in response to genistein and vehicle treatment. *=p<0.05 vs vehicle treated mdx mice.

FIG. 9 is a graph showing the strength to body weight ratio of mdx mice in response to genistein and vehicle treatment. *=p<0.05 vs vehicle treated mdx mice.

FIG. 10 is a graph showing fatigue levels of mdx mice in response to genistein and vehicle treatment.

FIG. 11 is a graph showing the creatine kinase activity of mdx mice in response to genistein and vehicle treatment. *=p<0.05 vs vehicle treated mdx mice.

FIG. 12 is a graph showing the level of necrosis in the biceps of mdx mice in response to genistein and vehicle treatment. *=p<0.05 vs vehicle treated mdx mice.

FIG. 13 is a graph showing the level of regeneration in the biceps of mdx mice in response to genistein and vehicle treatment. *=p<0.05 vs vehicle treated mdx mice.

FIG. 14 is a graph showing numbers of myogenin positive nuclei in mdx mice in response to genistein and vehicle treatment. *=p<0.05 vs vehicle treated mdx mice.

FIG. 15 is a graph showing the level of NF-κB activation of mdx mice in response to genistein and vehicle treatment. *=p<0.05 vs vehicle treated mdx mice. The figure also includes a representative autoradiogram.

FIG. 16 is a graph showing the level of maximal isometric force in muscles of mdx mice in response to genistein and vehicle treatment. *=p<0.05 vs vehicle treated mdx mice.

### DETAILED DESCRIPTION OF THE INVENTION

Improved methods have been developed for treating muscular dystrophies, especially Duchenne muscular dystrophy, with flavonoids.

Studies have shown that green tea extract and its component epigallocatechin gallate have a beneficial effect on mdx mice. It has now been discovered that specific types of flavonoids can be surprisingly effective at treating muscular dystrophies, especially Duchenne muscular dystrophy.

The present invention may be understood more readily by reference to the following detailed description of preferred embodiments of the invention and the non-limiting Examples included therein.

### The Flavonoids

The flavonoids used in various embodiments of the invention are based upon the following general structures, referred to herein as the (1) flavan nucleus and (2) isoflavan nucleus:

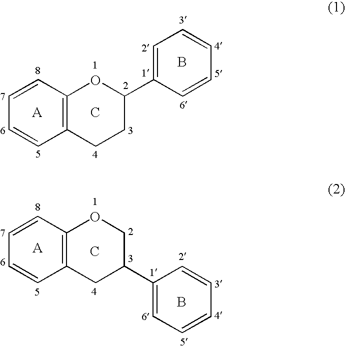

In embodiments wherein the flavonoid is based on the flavan nucleus, the carbons located at positions 8, 7, 6, 5, 4, 3, 2′, 3′, 4′, 5′, and/or 6′ may be modified by functional groups independently selected from the set consisting of —H, —OH, —SH, OR, —SR, —NH₂, —NHR, —NR₂, —NR₃⁺X⁻, a carbon, oxygen, nitrogen or sulfur, glycoside of a single or a combination of multiple sugars including, but not limited to aldopentoses, methyl-aldopentose, aldohexoses, ketohexose and their chemical derivatives thereof; wherein R is an alkyl group having between 1-10 carbon atoms; and X is selected from the group of pharmaceutically acceptable counter anions including, but not limited to hydroxyl, chloride, iodide, sulfate, phosphate, acetate, fluoride, and carbonate. In particularly desirable embodiments, the carbons may be modified by functional groups independently selected from the set consisting of —H, alcohol (—OH), ketone (═O), aldehyde (—CHO), carboxyl (—COOH), and glycosides of a single sugar. In other desirable embodiments, the carbons may be modified with alcohol groups (—OH) only. In yet another desirable embodiment, the carbon atoms of the “B” ring of the flavonoid have no substituted groups.

Similarly, in embodiments wherein the flavonoid is based on the isoflavan nucleus, the carbons located at positions 8, 7, 6, 5, 4, 2, 2′, 3′, 4′, 5′, and/or 6′ may be modified by functional groups independently selected from the set consisting of —H, —OH, —SH, OR, —SR, —NH₂, —NHR, —NR₂, —NR₃⁺X⁻, a carbon, oxygen, nitrogen or sulfur, glycoside of a single or a combination of multiple sugars including, but not limited to aldopentoses, methyl-aldopentose, aldohexoses, ketohexose and their chemical derivatives thereof; wherein R is an alkyl group having between 1-10 carbon atoms; and X is selected from the group of pharmaceutically acceptable counter anions including, but not limited to hydroxyl, chloride, iodide, sulfate, phosphate, acetate, fluoride, and carbonate. In particularly desirable embodiments, the carbons may be modified by functional groups independently selected from the set consisting of —H, alcohol (—OH), ketone (═O), aldehyde (—CHO), carboxyl (—COOH), and glycosides of a single sugar. In other desirable embodiments, the carbons only may be modified with alcohol groups (—OH). In yet another desirable embodiment, the carbon atoms of the “B” ring of the flavonoid have no substituted groups.

Flavones/Isoflavones

The flavonoid used in various embodiments of the invention may comprise a flavone or isoflavone, i.e. a flavonoid in which at least one carbon is modified with a ketone alcohol group. In embodiments wherein the flavonoid is a flavone, the carbons located at positions 8, 7, 6, 5, 4, 3, 2′, 3′, 4′, 5′, and/or 6′ may be modified. In embodiments wherein the flavonoid is an isoflavone, the carbons located at positions 8, 7, 6, 5, 4, 2, 2′, 3′, 4′, 5′, and/or 6′ may be modified.

In particularly desirable embodiments, the flavonoids used in various embodiments of the invention are based upon the following general structures, referred to herein as the (1) flav-4-one nucleus and (2) isoflav-4-one nucleus:

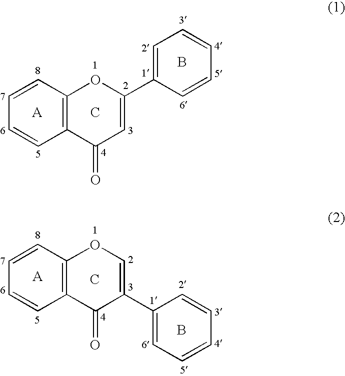

In embodiments wherein the flavonoid is based on the flav-4-one nucleus, the carbons located at positions 8, 7, 6, 5, 3, 2′, 3′, 4′, 5′, and/or 6′ may be modified by functional groups independently selected from the set consisting of —H, —OH, —SH, OR, —SR, —NH₂, —NHR, —NR₂, —NR₃⁺X⁻, a carbon, oxygen, nitrogen or sulfur, glycoside of a single or a combination of multiple sugars including, but not limited to aldopentoses, methyl-aldopentose, aldohexoses, ketohexose and their chemical derivatives thereof; wherein R is an alkyl group having between 1-10 carbon atoms; and X is selected from the group of pharmaceutically acceptable counter anions including, but not limited to hydroxyl, chloride, iodide, sulfate, phosphate, acetate, fluoride, and carbonate. In particularly desirable embodiments, the carbons may be modified by functional groups independently selected from the set consisting of —H, alcohol (—OH), ketone (═O), aldehyde (—CHO), carboxyl (—COOH), and glycosides of a single sugar. In other desirable embodiments, the carbons only may be modified with alcohol groups (—OH). In yet another desirable embodiment, the carbon atoms of the “B” ring of the flavonoid have no substituted groups.

Similarly, in embodiments wherein the flavonoid is based on the isoflav-4-one nucleus, the carbons located at positions 8, 7, 6, 5, 2, 2′, 3′, 4′, 5′, and/or 6′ may be modified by functional groups independently selected from the set consisting of —H, —OH, —SH, OR, —SR, —NH₂, —NHR, —NR₂, —NR₃⁺X⁻, a carbon, oxygen, nitrogen or sulfur, glycoside of a single or a combination of multiple sugars including, but not limited to aldopentoses, methyl-aldopentose, aldohexoses, ketohexose and their chemical derivatives thereof; wherein R is an alkyl group having between 1-10 carbon atoms; and X is selected from the group of pharmaceutically acceptable counter anions including, but not limited to hydroxyl, chloride, iodide, sulfate, phosphate, acetate, fluoride, and carbonate. In particularly desirable embodiments, the carbons may be modified by functional groups independently selected from the set consisting of —H, alcohol (—OH), ketone (═O), aldehyde (—CHO), carboxyl (—COOH), and glycosides of a single sugar. In other desirable embodiments, the carbons only may be modified with alcohol groups (—OH). In yet another desirable embodiment, the carbon atoms of the “B” ring of the flavonoid have no substituted groups.

In those embodiments were the flavonoid is based on the isoflav-4-one nucleus, it may be particularly desirable that one or more of the carbons located at positions 8, 7, 6, 5, 2, 2′, 3′, 4′, 5′, and/or 6′ be modified by alcohol (—OH) groups. The total number of carbons modified by alcohol groups may be between one and seven, more desirably between two and five, and yet more desirably three.

In one embodiment, the flavonoid is based on the isoflav-4-one nucleus and has the carbons located at positions 7 and 4′ modified by alcohol (—OH) groups. Non-limiting examples of such isoflavones include daidzein (7-Hydroxy-3-(4-hydroxyphenyl) chromen-4-one) and glycitein (7-Hydroxy-3-(4-hydroxyphenyl)-6-methoxy-4-chroenone), the structures of which are illustrated below:

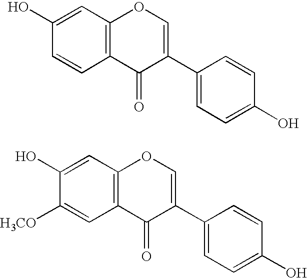

In a particularly desirable embodiment, the flavonoid is based on the isoflav-4-one nucleus and has the carbons located at positions 5, 7, and 4′ modified by alcohol (—OH) groups. A non-limiting example of such an isoflavone is genistein (5,7-Dihydroxy-3-(4-hydroxyphenyl)chromen-4-one), the structure of which is illustrated below:

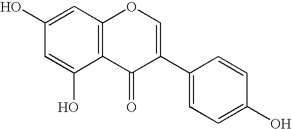

In yet another desirable embodiment, the flavonoid is a “glucoside form” of a flavone or isoflavone, i.e. a flavone or isoflavone in which at least one carbon is modified by a glycoside that is derived from glucose. Non-limiting examples of such glucoside forms include genistin (genistein-7-O-beta-D-glucopyranoside), daidzin (7-(−D-Glucopyranosyloxy)-3-(4-hydroxyphenyl)-4H-1-benzopyran-4-one), and glycitin (3-(4-Hydroxyphenyl)-6-methoxy-7-[(2S,3R,4S,5S,6R)-3,4,5-trihydroxy-6-(hydroxymethyl)oxan-2-yl]oxychromen-4-one). Following ingestion, these glucoside forms can be converted to aglycone form. For example, genistin can be converted into genistein, daidzein can be converted to daidzein, and glycitin can be converted to glycitein.

Flavanols/Isoflavanols

The flavonoid used in various embodiments of the invention may comprise a flavanol or isoflavanol, i.e. a flavonoid in which at least one carbon is modified with an alcohol group. In embodiments wherein the flavonoid is a flavanol, the carbons located at positions 8, 7, 6, 5, 4, 3, 2′, 3′, 4′, 5′, and/or 6′ may be modified. In embodiments wherein the flavonoid is an isoflavanol, the carbons located at positions 8, 7, 6, 5, 4, 2, 2′, 3′, 4′, 5′, and/or 6′ may be modified.

In particularly desirable embodiments, the flavonoids used in various embodiments of the invention are based upon the following general structure, referred to herein as the flavan-3-ol nucleus:

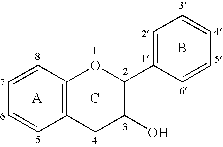

In embodiments wherein the flavonoid is based on the flavan-3-ol structure, the carbons located at positions 8, 7, 6, 5, 4, 2′, 3′, 4′, 5′, and/or 6′ may be modified by functional groups independently selected from the set consisting of —H, —OH, —SH, OR, —SR, —NH₂, —NHR, —NR₂, —NR₃⁺X⁻, a carbon, oxygen, nitrogen or sulfur, glycoside of a single or a combination of multiple sugars including, but not limited to aldopentoses, methyl-aldopentose, aldohexoses, ketohexose and their chemical derivatives thereof; wherein R is an alkyl group having between 1-10 carbon atoms; and X is selected from the group of pharmaceutically acceptable counter anions including, but not limited to hydroxyl, chloride, iodide, sulfate, phosphate, acetate, fluoride, and carbonate. In particularly desirable embodiments, the carbons may be modified by functional groups independently selected from the set consisting of —H, alcohol (—OH), ketone (═O), aldehyde (—CHO), carboxyl (—COOH), and glycosides of a single sugar. In other desirable embodiments, the carbons only may be modified with alcohol groups (—OH). In yet another desirable embodiment, the carbon atoms of the “B” ring of the flavonoid have no substituted groups.

In those embodiments were the flavonoid is based on the flavan-3-ol structure, it may be particularly desirable that one or more of the carbons located at positions 8, 7, 6, 5, 4, 2′, 3′, 4′, 5′, and/or 6′ be modified by alcohol (—OH) groups. The total number of carbons modified by alcohol groups may be between one and seven, more desirably between three and six, and yet more desirably five.

In a particularly desirable embodiment, the flavonoid is based on the flavan-3-ol structure and has the carbons located at positions 3, 3′, 4′, 5, and 7 modified by alcohol (—OH) groups. Non-limiting examples of a such a flavonoid include catechin and its stereoisomer epicatechin (3,3′,4′,5,7-pentahydroxyflavan), the structure of which is illustrated below:

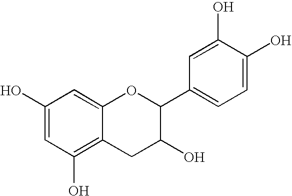

In a different embodiment, the flavonoid is an isoflavanol and has the carbons located at positions 7 and 4′ modified by alcohol (—OH) groups. A non-limiting example of a such a flavonoid is equol ((S)-3,4-Dihydro-3-(4-hydroxyphenyl)-2H-1-benzopyran-7-ol), the structure of which is illustrated below:

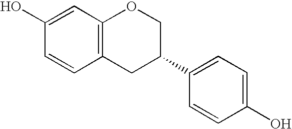

In yet another embodiment, the flavanol or isoflavanol is in polymeric form. Non-limiting examples of suitable polymeric flavanols and isoflavanols include proanthocyanidin polymers such as polymers of catechin and epicatechin, polymers of anthocyanins, and polymers of gallate salts.

Free-B-Ring Flavonoids

The flavonoid used in various embodiments of the invention may comprise a Free-B-Ring flavonoid or Free-B-Ring isoflavonoid, i.e. a flavonoid in which none of the carbons on the aromatic B-ring are modified. Free-B-Ring flavonoids are relatively rare. Out of a total 9396 flavonoids synthesized or isolated from natural sources, only 231 Free-B-Ring flavonoids are known (See ref 40 below).

In particularly desirable embodiments, the flavonoids used in various embodiments of the invention are based upon the following general structures, referred to herein as the (1) Free-B-Ring flavonoid nucleus and (2) Free-B-Ring isoflavonoid nucleus:

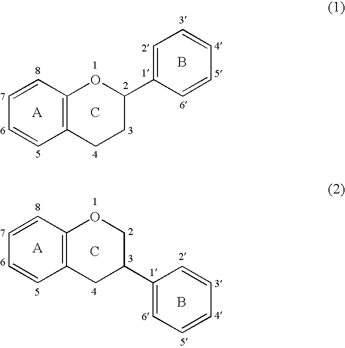

In embodiments wherein the flavonoid is based on the Free-B-Ring flavonoid nucleus, the carbons located at positions 8, 7, 6, 5, 4, and/or 3 may be modified by functional groups independently selected from the set consisting of —H, —OH, —SH, OR, —SR, —NH₂, —NHR, —NR₂, —NR₃⁺X⁻, a carbon, oxygen, nitrogen or sulfur, glycoside of a single or a combination of multiple sugars including, but not limited to aldopentoses, methyl-aldopentose, aldohexoses, ketohexose and their chemical derivatives thereof; wherein R is an alkyl group having between 1-10 carbon atoms; and X is selected from the group of pharmaceutically acceptable counter anions including, but not limited to hydroxyl, chloride, iodide, sulfate, phosphate, acetate, fluoride, and carbonate. In particularly desirable embodiments, the carbons may be modified by functional groups independently selected from the set consisting of —H, alcohol (—OH), ketone (═O), aldehyde (—CHO), carboxyl (—COOH), and glycosides of a single sugar.

Similarly, in embodiments wherein the flavonoid is based on the Free-B-Ring isoflavonoid nucleus, the carbons located at positions 8, 7, 6, 5, 4, and/or 2 may be modified by functional groups independently selected from the set consisting of —H, —OH, —SH, OR, —SR, —NH₂, —NHR, —NR₂, NR₃⁺X⁻, a carbon, oxygen, nitrogen or sulfur, glycoside of a single or a combination of multiple sugars including, but not limited to aldopentoses, methyl-aldopentose, aldohexoses, ketohexose and their chemical derivatives thereof; wherein R is an alkyl group having between 1-10 carbon atoms; and X is selected from the group of pharmaceutically acceptable counter anions including, but not limited to hydroxyl, chloride, iodide, sulfate, phosphate, acetate, fluoride, and carbonate. In particularly desirable embodiments, the carbons may be modified by functional groups independently selected from the set consisting of —H, alcohol (—OH), ketone (═O), aldehyde (—CHO), carboxyl (—COOH), and glycosides of a single sugar.

In particularly desirable embodiments, the Free-B-Ring flavanoids used in various embodiments of the invention are based upon the following general structures, referred to herein as the (1) Free-B-Ring flavone nucleus and (2) Free-B-Ring isoflavone nucleus:

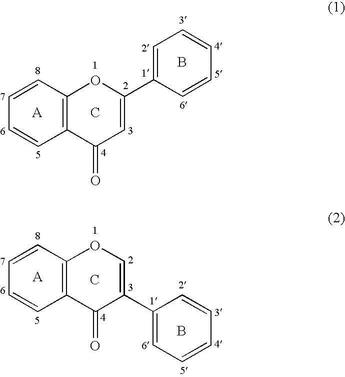

In embodiments wherein the flavonoid is based on the Free-B-Ring flavone nucleus, the carbons located at positions 8, 7, 6, 5, and/or 3 may be modified by functional groups independently selected from the set consisting of —H, —OH, —SH, OR, —SR, —NH₂, —NHR, —NR2, —NR3+X−, a carbon, oxygen, nitrogen or sulfur, glycoside of a single or a combination of multiple sugars including, but not limited to aldopentoses, methyl-aldopentose, aldohexoses, ketohexose and their chemical derivatives thereof; wherein R is an alkyl group having between 1-10 carbon atoms; and X is selected from the group of pharmaceutically acceptable counter anions including, but not limited to hydroxyl, chloride, iodide, sulfate, phosphate, acetate, fluoride, and carbonate. In particularly desirable embodiments, the carbons may be modified by functional groups independently selected from the set consisting of —H, alcohol (—OH), ketone (═O), aldehyde (—CHO), carboxyl (—COOH), and glycosides of a single sugar. In other desirable embodiments, the carbons only may be modified with alcohol groups (—OH).

Similarly, in embodiments wherein the flavonoid is based on the Free-B-Ring isoflavone nucleus, the carbons located at positions 8, 7, 6, 5, and/or 2 may be modified by functional groups independently selected from the set consisting of —H, —OH, —SH, OR, —SR, —NH2, —NHR, —NR2, —NR3+X−, a carbon, oxygen, nitrogen or sulfur, glycoside of a single or a combination of multiple sugars including, but not limited to aldopentoses, methyl-aldopentose, aldohexoses, ketohexose and their chemical derivatives thereof; wherein R is an alkyl group having between 1-10 carbon atoms; and X is selected from the group of pharmaceutically acceptable counter anions including, but not limited to hydroxyl, chloride, iodide, sulfate, phosphate, acetate, fluoride, and carbonate. In particularly desirable embodiments, the carbons may be modified by functional groups independently selected from the set consisting of —H, alcohol (—OH), ketone (═O), aldehyde (—CHO), carboxyl (—COOH), and glycosides of a single sugar. In other desirable embodiments, the carbons only may be modified with alcohol groups (—OH).

In those embodiments were the flavonoid is based on the Free-B-Ring flavone nucleus, it may be particularly desirable that one or more of the carbons located at positions 8, 7, 6, 5, and/or 3 be modified by alcohol (—OH) groups. The total number of carbons modified by alcohol groups may be between one and four, and more desirably two. In a particularly desirable embodiment, the flavonoid is based on the Free-B-Ring flavone and has carbons located at positions 6 and 5 modified by alcohol groups.

Furthermore, in those embodiments were the flavonoid is based on the Free-B-Ring flavone nucleus, one or more of the carbons located at positions 8, 7, 6, 5, and/or 3 be modified by a glycoside group. In certain embodiments, the glycoside is a single sugar, non-limiting examples of which include glucose (dextrose), fructose, galactose, xylose and ribose. The glycoside may have a pyranose structure (i.e. a chemical structure that includes a six-membered ring consisting of five carbons and one oxygen). In desirable embodiments, the carbon at the 7 position is modified with a glycoside group with a pyranose structure.

In a particularly desirable embodiment, the flavonoid is based on the Free-B-Ring flavone nucleus, and has carbons located at positions 7, 5, and 3 modified by alcohol (—OH) groups. A non-limiting example of a such a Free-B-Ring flavonoid is galangin (3,5,7-trihydroxyflavone), the structure of which is illustrated below (see also Ref 41-45):

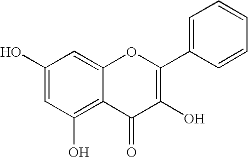

In another particularly desirable embodiment, the flavonoid is based on the Free-B-Ring flavone nucleus, and has carbons located at positions 7, 6, and 5 modified by alcohol (—OH) groups. A non-limiting example of a such a Free-B-Ring flavonoid is baicalein (5,6,7-trihydroxyflavone), the structure of which is illustrated below (see also Ref 46):

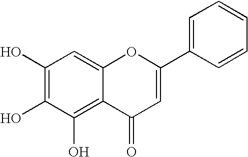

In yet another particularly desirable embodiment, the flavonoid is based on the Free-B-Ring flavone nucleus, has carbons located at positions 6 and 5 modified by alcohol (—OH) groups, and has the carbon at the 7 position modified with a glycoside group with a pyranose structure. A non-limiting example of a such a Free-B-Ring flavonoid is baicalin (5,6,7-trihydroxyflavone,7-O-β-D-glucuronopyranoside), the structure of which is illustrated below:

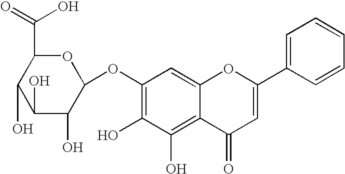

### The Methods of Treating Muscular Dystrophies

The methods described below can be used to treat muscular dystrophies, especially Duchenne muscular dystrophy. As used herein, the term “muscular dystrophies” refers to the group of genetic, hereditary muscle diseases often characterized by progressive skeletal muscle weakness, defects in muscle proteins, and the death of muscle cells and tissue. Examples of muscular dystrophy diseases include Duchenne, Becker, limb girdle, congenital, facioscapulohumeral, myotonic, oculopharyngeal, distal, and Emery-Dreifuss muscular dystrophies. In a particular embodiment, the methods can be used to treat Duchenne muscular dystrophy (DMD).

Methods of Treatment with Isoflavones

In one embodiment, the method of treating muscular dystrophies, especially Duchenne muscular dystrophy, comprises (a) providing a pharmaceutical composition comprising a therapeutically effective amount of flavonoid, and (b) administering the composition to a human patient, wherein the flavonoid comprises an isoflav-4-one nucleus, and wherein at least one of the carbons of the nucleus located at positions 8, 7, 6, 5, 2, 2′, 3′, 4′, 5′, or 6′ is modified by an alcohol group.

Desirably, the number of carbons of the isoflav-4-one nucleus modified by alcohol groups is between two and five, and more desirably three. Even more desirably, the carbons at positions 5, 7, and 4′ are modified by alcohol groups. In particular embodiments, the carbons located at positions 8, 7, 6, 5, 2, 2′, 3′, 4′, 5′, and 6′ are not modified by any functional groups that are not alcohol groups. In a particularly preferred embodiment, the flavonoid comprises genistein.

The step of administering the composition can comprise essentially any method of administration known in the art. In one embodiment, step of administering the composition comprises enteral administration. In another embodiment, the step of administering the composition comprises parenteral administration (for example, subcutaneous, intravenous, or intraperitoneal injection). In yet another embodiment, the step of administering the composition comprises topical administration.

The step of administering the composition, particularly when administered orally, can comprise administering the flavonoid in an amount in the range of about 100 mg to about 2000 mg per day, preferably from about 150 to about 1,500 mg per day, most preferably from about 250 mg to about 1000 mg per day. When administered parenterally, the step of administering the composition can comprise administering the flavonoid in an amount in the range of about 4.0 to 350 mg per day, 7.5 to 200 mg per day, or 10.0 to 100 mg per day.

Methods of Treatment with Flavanols

In one embodiment, the method of treating muscular dystrophies, especially Duchenne muscular dystrophy, comprises (a) providing a pharmaceutical composition comprising a therapeutically effective amount of flavonoid, and (b) administering the composition to a human patient, wherein the flavonoid comprises a flavan-3-ol nucleus, and wherein at least one of the carbons of the nucleus located at positions 8, 7, 6, 5, 4, 2′, 3′, 4′, 5′, or 6′ is modified by an alcohol group.

Desirably, the number of carbons of the flavan-3-ol nucleus modified by alcohol groups is between two and six, and more desirably four. Even more desirably, the carbons at positions 5, 7, 3′, and 4′ are modified by alcohol groups. In particular embodiments, the carbons located at positions 8, 6, 4, 2′, 5′, and 6′ are not modified by any functional groups that are not alcohol groups. In a particularly preferred embodiment, the flavonoid comprises at least one of catechin or epicatechin.

The step of administering the composition can comprise essentially any method of administration known in the art. In one embodiment, step of administering the composition comprises enteral administration. In another embodiment, the step of administering the composition comprises parenteral administration (for example, subcutaneous, intravenous, or intraperitoneal injection). In yet another embodiment, the step of administering the composition comprises topical administration.

The step of administering the composition, particularly when administered orally, can comprise administering the flavonoid in an amount in the range of about 100 mg to about 2000 mg per day, preferably from about 150 to about 1,500 mg per day, most preferably from about 250 mg to about 1000 mg per day. When administered parenterally, the step of administering the composition can comprise administering the flavonoid in an amount in the range of about 4.0 to 350 mg per day, 7.5 to 200 mg per day, or 10.0 to 100 mg per day.

Methods of Treatment with Free-B-Ring Flavones

In one embodiment, the method of treating muscular dystrophies, especially Duchenne muscular dystrophy, comprises (a) providing a pharmaceutical composition comprising a therapeutically effective amount of flavonoid, and (b) administering the composition to a human patient, wherein the flavonoid comprises a Free-B-Ring flavone nucleus, wherein at least one of the carbons of the nucleus located at positions 8, 7, 6, 5 or 3 is modified by an alcohol group, and wherein at least one of the carbons of the nucleus located at positions 8, 7, 6, 5 or 3 is modified by a glycoside group.

Desirably, the number of carbons of the Free-B-Ring flavone nucleus modified by alcohol groups is between one and three, and more desirably two. Even more desirably the carbons at positions 5 and 6 are modified by alcohol groups. In particular embodiments, the carbons located at positions 8, 7, 6, 5 and 3 are not modified by any functional groups that are not alcohol or glycoside groups.

It also is desirable that the glycoside group comprises a glycoside of a single sugar, and more desirable that the glycoside group comprises a pyranose structure. Even more desirably, the carbon at position 7 is modified with a glycoside of a single sugar.

The glycoside can comprise a pyranose structure. In a particular embodiment, the carbons at positions 5 and 6 are modified by alcohol groups, and the carbon at position 7 is modified with a pyranose structure. In a particularly preferred embodiment, the flavonoid comprises baicalin.

The step of administering the composition can comprise essentially any method of administration known in the art. In one embodiment, step of administering the composition comprises enteral administration. In another embodiment, the step of administering the composition comprises parenteral administration (for example, subcutaneous, intravenous, or intraperitoneal injection). In yet another embodiment, the step of administering the composition comprises topical administration.

The step of administering the composition, particularly when administered orally, can comprise administering the flavonoid in an amount in the range of about 100 mg to about 2000 mg per day, preferably from about 150 to about 1,500 mg per day, most preferably from about 250 mg to about 1000 mg per day. When administered parenterally, the step of administering the composition can comprise administering the flavonoid in an amount in the range of about 4.0 to 350 mg per day, 7.5 to 200 mg per day, or 10.0 to 100 mg per day.

Methods of Treatment with Free-B-Ring Flavones and Flavanols/Isoflavanols

In one embodiment, the method of treating muscular dystrophies, especially Duchenne muscular dystrophy, comprises (a) providing a pharmaceutical composition comprising a therapeutically effective amount of a first flavonoid and a therapeutically effective amount of second flavonoid, and (b) administering the composition to a human patient, wherein the first flavonoid comprises a Free-B-Ring flavone nucleus, wherein at least one of the carbons of the nucleus located at positions 8, 7, 6, 5 or 3 is modified by an alcohol group, and wherein at least one of the carbons of the nucleus located at positions 8, 7, 6, 5 or 3 is modified by a glycoside group; and wherein the second flavonoid comprises a flavan-3-ol nucleus, wherein at least one of the carbons of the flavan-3-ol nucleus located at positions 8, 7, 6, 5, 4, 2′, 3′, 4′, 5′, or 6′ is modified by an alcohol group.

The first flavonoid can comprise any Free-B-Ring flavone as described in the Methods of Treatment with Free-B-Ring Flavones above. In a particularly preferred embodiment, the first flavonoid comprises baicalin. The second flavonoid can comprise any flavan-3-ol as described in the Methods of Treatment with Flavanols above. In a particularly preferred embodiment, the second flavonoid comprises at least one of catechin or epicatechin.

The step of administering the composition can comprise essentially any method of administration known in the art. In one embodiment, the step of administering the composition comprises enteral administration. In another embodiment, the step of administering the composition comprises parenteral administration.

The step of administering the composition, particularly when administered orally, can comprise administering the flavonoid in an amount in the range of about 100 mg to about 2000 mg per day, preferably from about 150 to about 1,500 mg per day, most preferably from about 250 mg to about 1000 mg per day. When administered parenterally, the step of administering the composition can comprise administering the flavonoid in an amount in the range of about 4.0 to 350 mg per day, 7.5 to 200 mg per day, or 10.0 to 100 mg per day.

All of the foregoing embodiments can also be defined with reference to the ratio of first and second flavonoids, when more than one flavonoid is administered. The ratio of the amount of the first flavonoid to the amount of the second flavonoid in the pharmaceutical composition can be in the range of about 99:1 to about 1:99, in the range of about 90:10 to about 10:90, in the range of about 75:25 to about 25:75, or in the range of about 60:40 to about 40:60, in various embodiments.

### Example 1

### Treatment of Wild-Type and MDX Mice with Flavocoxid

### Materials and Methods

Animals

Male mdx and wild-type C57BJ/10 (WT) mice were obtained from Jackson Laboratories (Bar Harbor, Me., USA). Mice were housed in plastic cages in a temperature-controlled environment with a 12-h light/dark cycle and access to standard laboratory food and tap water. The investigation conformed to the Guide for the Care and Use of Laboratory Animals published by the US National Institutes of Health (NIH Publication No. 85-23, revised 1996).

Five-week old mdx and WT mice were treated for 5 weeks with daily 100 μl intraperitoneal injections with either flavocoxid (a mixture of catechin and baicalin) (n=8, 5 mg/kg), methylprednisolone (n=8, 0.75 mg/Kg) or vehicle (n=8, 33% (v/v) dimethylsulphoxide (DMSO) in 0.9% NaCl). At the end of the experiments, animals were anaesthetized with intraperitoneal administration of sodium penthobarbital (80 mg/kg). Blood was then collected by intracardiac puncture and used to analyze creatine kinase (CK) levels. The biceps, quadriceps and extensor digitorum longus (EDL) muscles were removed bilaterally and immediately frozen in liquid nitrogen-cooled isopentane and stored at −80° C. for histological and biochemical evaluations.

Drug

Flavocoxid (Limbrel®) was from Primus Pharmaceuticals, Inc (Scottsdale, Ariz., USA). Methylprednisolone was purchased by Sigma Aldrich (Milan, Italy). All substances were prepared fresh daily and administered in a volume of 100 μl per animal.

Statistical Analysis

Results were expressed as mean±SD. Statistical evaluation was performed by using one-way analysis of variance followed by Dunnett's post hoc tests and paired Student's t-test with the use of the InPlotPrism software version 3.0 (GraphPad Software, San Diego, USA). P values of <0.05 were considered significant.

### Results

Strength Examinations

Mice were weighed and examined for forelimb strength at baseline and after 5 weeks of treatment. Strength testing consisted of five separate measurements using a grip meter attached to a force transducer that measures peak force generated (Stoelting Co, Wooddale Ill., USA). Briefly, the mouse grabed the trapeze bar as it was pulled backward and the peak pull force in grams was recorded on a display upon release by the animal. The three highest measurements for each animal were averaged to give the strength score. The degree of fatigue was calculated by comparing the first two pulls to the last two pulls. The decrement between pulls 1+2 and pulls 4+5 gave the measure of fatigue.

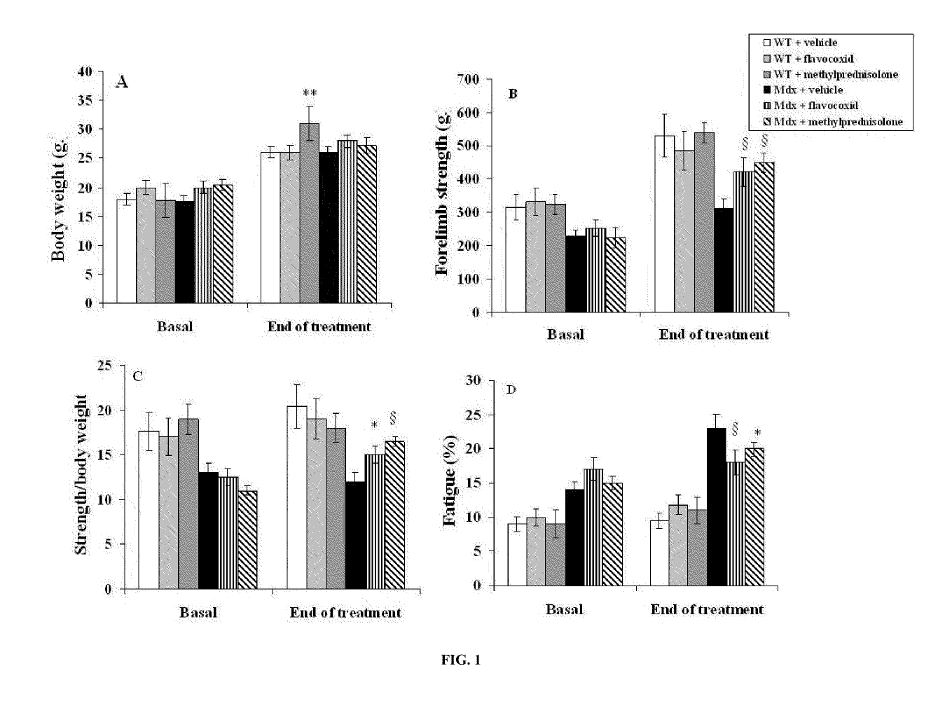

Body weight was not significantly different among the animal groups at baseline. As shown in FIG. 1A, the only statistically significant weight difference was that methylprednisolone treated WT mice showed an increase in body weight as compared to WT mice treated with vehicle (p<0.01). Comparing the values longitudinally, at the end of the experiment all groups had an increased body weight (p<0.05) (FIG. 1A).

As shown in FIG. 1B and FIG. 1C, baseline strength and strength normalized to weight was significantly lower in mdx mice groups as compared to WT groups (p<0.05). At the end of treatment, flavocoxid-treated and methylprednisolone-treated mdx mice had higher forelimb strength (respectively, +26%; p<0.01 and +30%; p<0.01) and strength normalized to weight (+20%; p<0.01 and +27%; p<0.01) compared to vehicle-treated mdx mice. In all groups, the forelimb strength increased as compared with baseline values (p<0.01 in all groups) (FIG. 1B). However, when normalized to weight, only the flavocoxid and methylprednisolone-treated mdx mice showed a statistically significant increase in strength (respectively, p<0.05 and p<0.01) (FIG. 1C).

As shown in FIG. 1D, baseline fatigue percentage was significantly higher in mdx mice groups as compared to WT groups (p<0.01). After treatment, the three mdx groups had a higher level of fatigue as compared to WT groups (p<0.001 in mdx+vehicle; p<0.01 in mdx+flavocoxid and mdx+methylprednisolone). However, the fatigue levels were significantly lower in flavocoxid-treated (−22%; p<0.01) and methylprednisolone-treated (−13%; p<0.05) mdx mice as compared to vehicle-treated mdx. Furthermore, the percentage of fatigue increased more in the vehicle-treated mdx mice (p<0.01) than in the methylprednisolone-treated mdx mice (p<0.05), and remained stable in the flavocoxid-treated mdx mice (FIG. 1D). No treatments resulted in statistically significant changes in the forelimb strength, strength normalized to body weight, or fatigue levels of the WT animals.

Serum Creatine Kinase (CK) Evaluation

Blood samples were centrifuged at 6,000 RPM and the serum stored at −80° C. until the day of analysis. Serum CK was evaluated at 37° C. using a commercially available kit (Randox Laboratories LTD, Crumlin, Co. Antrim U.K). The results were expressed as U/L.

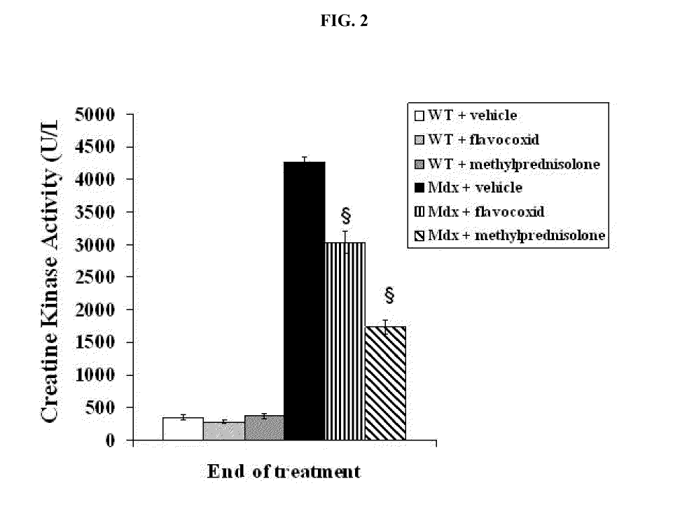

As shown in FIG. 2, low CK levels were observed in WT animals treated either with vehicle, flavocoxid or methylprednisolone (WT+vehicle=353±33 U/L; WT+Flavocoxid=280±28 U/L; WT+methylprednisolone=365±28 U/L). Vehicle treated mdx mice showed a significant increase in serum CK levels (mdx+vehicle=4276±79 U/L; p<0.01 vs WT+vehicle). Importantly, treatment with either flavocoxid or methylprednisolone resulted in a statistically significant reduction of the CK levels in mdx mice (mdx+flavocoxid=3030±173 U/L and mdx+methylprednisolone=1737±103 U/L; p<0.01 and p<0.001 vs mdx+vehicle).

Histological Studies

A 10-μm-thick transverse cryostat section was obtained from the midpoint of the biceps muscle body. The whole muscle cross-sections (corresponding to a mean area of 2.51 mm²) were stained with hematoxylin-eosin (H&E) and were examined by a blinded observer, using the AxioVision 2.05 image analysis system equipped with Axiocam camera scanner (Zeiss, Munchen, Germany). The following four areas were recognized with patchy distribution: (i) normal fibers, identified by the presence of peripheral nuclei; (ii) centrally nucleated fibers, identified by normal size but with central nuclei; (iii) regenerating fibers, identified by small size, basophilic cytoplasm and central nuclei; (iv) necrotic fibers, identified by pale cytoplasm and phagocytosis. The results were expressed as the ratio of the area occupied by normal fibers, centrally nucleated fibers, regenerating fibers or necrotic fibers divided by the total surface area as a percentage.

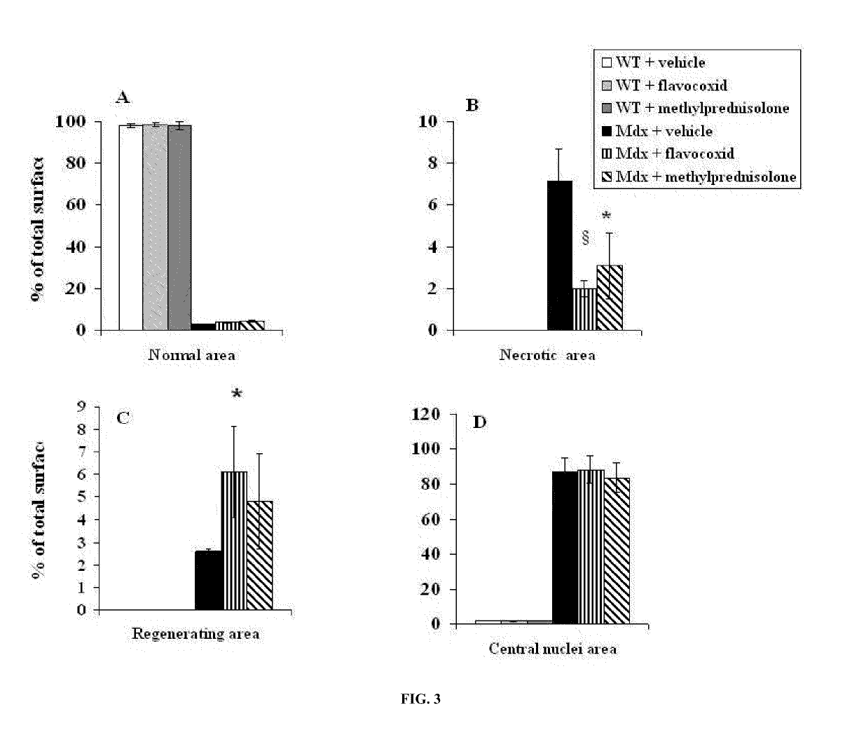

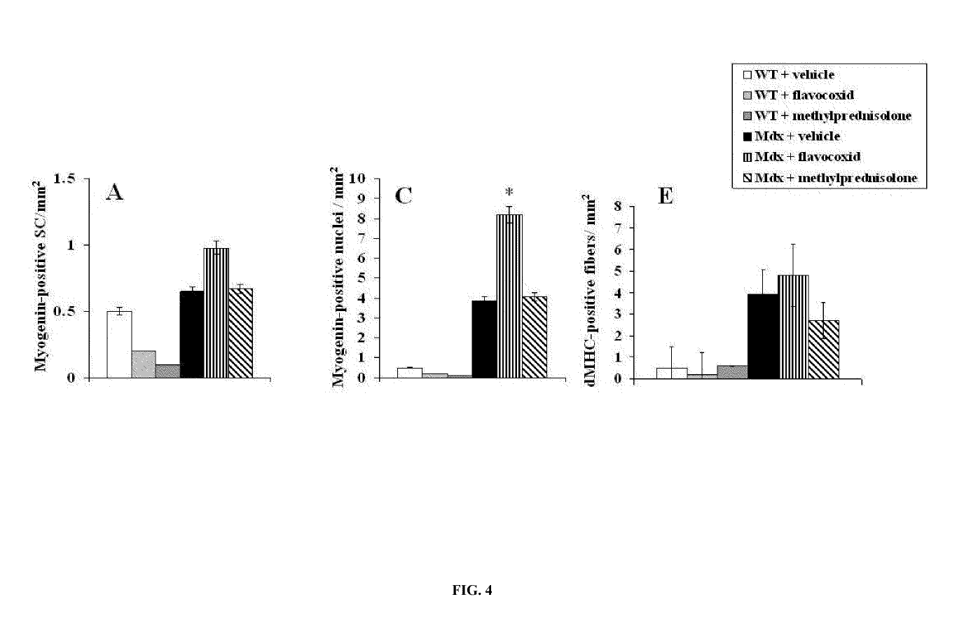

As shown in FIG. 3, the wild type animals had normal biceps muscles that were not modified by treatment with flavocoxid and methylprednisolone. Biceps muscles from vehicle-treated mdx mice showed both necrosis and regeneration (FIGS. 3B, 3C, and 4A). Quantitative morphological evaluation of biceps muscle from flavocoxid-treated mdx mice showed a statistically significant decrease in necrotic area (p<0.01) and a statistically significant increase in regenerating area (p<0.05) (FIGS. 3B, 3C, and 4B). Likewise, methylprednisolone-treated biceps of mdx mice revealed a decrease in necrotic area (p<0.01) and an increase in regenerating area (FIGS. 3B, 3C, and 4C).

Immunohistochemistry

Immunohistochemical detection of specific antigens was performed on 7-μm-thick transverse cryostat sections from biceps muscles. A rabbit polyclonal antibody against myogenin (1:60; sc-576, Santa Cruz Biotechnology, Inc., Santa Cruz, Calif.) and a mouse monoclonal antibody against developmental myosin heavy chain (dMHC) (1:20; NCL-MHCd, Novocastra Laboratories Ltd, Newcastle upon Tyne, UK) were used to study regeneration. Myogenin is expressed by differentiating myoblasts and is considered to be an ‘early’ marker of regeneration. DMHC was used as a marker of ‘later’ regeneration. Visualization of antibody staining was achieved using a peroxidase detection system (ABComplex/HRP kit, Dako, Milan, Italy) and the 3,3-diaminobenzidine tetrahydrochloride as chromogen. In the case of dMHC, the AEC substrate chromogen (Zymed Laboratories, San Francisco, Calif.) was used for color development.

As shown in FIG. 4, wild type animals showed a very low number of myogenin-positive nuclei and satellite cells (SC) per mm² and dMHC-positive fibers per mm² that was not modified by treatment with flavocoxid or methylprednisolone. Immunohistochemical analysis revealed a statistically significant increase in the number of myogenin-positive nuclei per mm² in flavocoxid-treated mdx mice as compared to vehicle-treated mdx mice (8.21 vs. 3.87 cells/mm²; P<0.05), and a trend towards an increase in the number of myogenin-positive SC per mm² and of dMHC-positive fibers per mm². Methylprednisolone treatment did not modify the number of myogenin-positive nuclei and SC per mm² or dMHC-positive fibers per mm² in mdx mice.

Electrophoretic Mobility Shift Assay (EMSA)

Nuclear proteins were isolated from approximately 50 mg of frozen skeletal muscle tissue (See ref 15 below). Twenty micrograms of nuclear extract was incubated for 30 min at room temperature with 50 fmol of biotin-end-labeled 45-mer double-strand NF-κB oligonucleotide from the HIV long terminal repeat, 5′-TTGTTACAAGGGACTTTCCGCTGGGGACTTTCCAGGAGGCGTGGG-3′ containing 2 (underlined) NF-κB binding sites. Both complimentary oligos were end-labeled separately and then annealed prior to use. Binding reaction mixtures were prepared in a final volume of 20 μL HEPES buffer containing 1 mg double-strand poly dI/dC, 10% glycerol, 100 mM MgCl2 and 1% Nonidet P-40, performed with the LightShift™ Chemiluminescent EMSA Kit (Pierce, Milan, Italy), according to the manufacturer's instructions. Bound complexes were separated on 7.5% nondenaturating polyacrylamide gels, blotted onto nylon membrane and visualized on Kodak X-ray film (Kodak, Milan, Italy) by autoradiography. The results were expressed as relative integrated intensity compared with normal controls, considering exposure time, background levels and known protein concentration of an Epstein Barr virus nuclear antigen-1 extract, which was used as EMSA control.

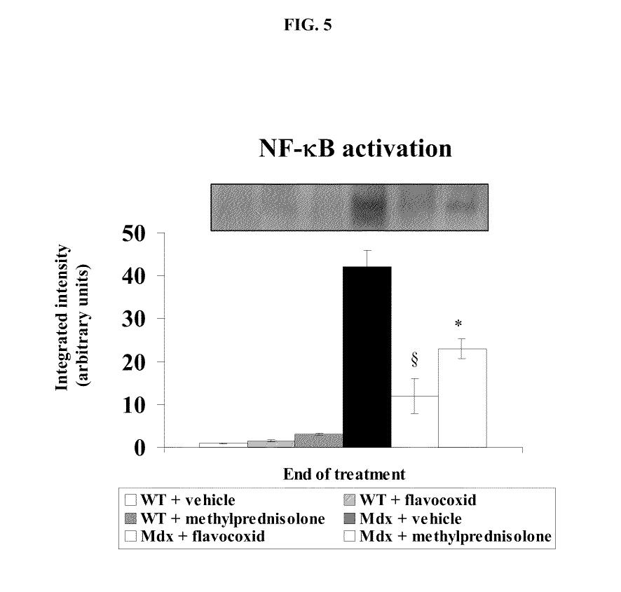

As shown in FIG. 5, NF-κB DNA binding activity was markedly increased in vehicle treated mdx mice as compared with WT mice (p<0.01). Furthermore, treatment with flavocoxid and methylprednisolone drastically reduced NF-κB binding activity in mdx mice (p<0.01).

Biochemical Assays (Hydrogen Peroxide and Total Peroxidase)

Thirty milligrams of muscle specimen was homogenized in 20 volumes of ice-cold buffer containing 0.5 M KCL, then centrifuged at 5500 g for 10 min. Protein concentration of tissue homogenates was determined by Lowry assay. An aliquot of 200 μl of muscle homogenate was used to perform hydrogen peroxide (HP) and peroxidase assays by spectrophotometry using Amplex Red Hydrogen Peroxide/peroxidase Assay kit (Molecular Probes Inc., Eugene, Oreg., USA).

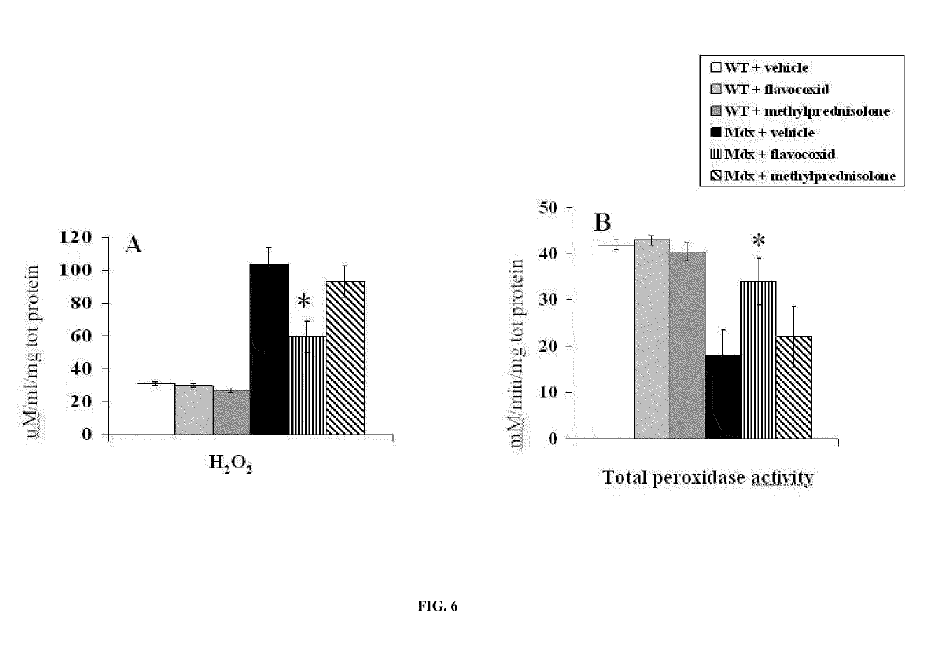

As shown in FIG. 6, H₂O₂ content and total peroxidase activity were unchanged in WT animals after flavocoxid or methylprednisolone treatment as compared with vehicle treated mice. Mdx mice showed markers of oxidative stress damage, characterized by a significant increase in the H₂O₂ content, accompanied by a concomitant decrease in activity of total peroxidase, when compared to WT animals. Flavocoxid administration in mdx mice resulted in a reduction of H₂O₂ level and an increase in the total peroxidase activity (p<0.01).

Isolation of Cytoplasmatic Proteins and Determination of COX-2,5-LOX, P-38, JNK and TNF-α

Muscle samples were homogenized in lysis buffer (1% Triton; 20 mM Tris/—HCl, pH 8.0; 137 mM NaCl; 10% glycerol; 5 mM EDTA; 1 mM phenylmethylsulfonyl fluoride; 1% aprotinin; 15 μg/mL leupeptin). Protein samples (30 μg) were denatured in reducing buffer (62 mmol Tris/HCl, pH 6.8, 10% glycerol; 2% SDS; 5% β-mercaptoethanol; 0.003% bromophenol blue) and separated by electrophoresis on a SDS (12%) polyacrylamide gel. The separated proteins were transferred on to a nitrocellulose membrane using the transfer buffer (39 mM glycine, 48 mM Tris/HCl pH 8.3, 20% methanol) at 200 mA for 1 h. The membranes were stained with Ponceau S (0.005% in 1% acetic acid) to confirm equal amounts of protein and blocked with 5% non fat dry milk in TBS-0.1% Tween for 1 h at room temperature, washed three times for 10 min each in TBS-0.1% Tween, and incubated with a primary phosphorylated antibody for COX-2 (Abcam, UK), 5-LOX (Abcam), phospho-p-38 (Cell Signaling, USA), phospho-JNK-1 (Cell Signaling, USA) and TNF-α (Chemicon, USA) in TBS-0.1% Tween overnight at 4° C. After being washed three times for 10 min each in TBS-0.1% Tween, the membranes were incubated with a specific peroxidase-conjugated secondary antibody (Pierce, UK) for 1 h at room temperature. After washing, the membranes were analyzed by the enhanced chemiluminescence system according to the manufacture's protocol (Amersham, UK). The protein signal was quantified by scanning densitometry using a bio-image analysis system (Bio-Profil Celbio, Italy). The results from each experimental group were expressed as relative integrated intensity compared with controls measured with the same batch. Equal loading of protein was assessed on stripped blots by immunodetection of β-actin with a rabbit monoclonal antibody (Cell Signaling, USA) and peroxidase-conjugated goat anti-rabbit immunoglobulin G (Pierce, UK). All antibodies were purified by protein A and peptide affinity chromatography.

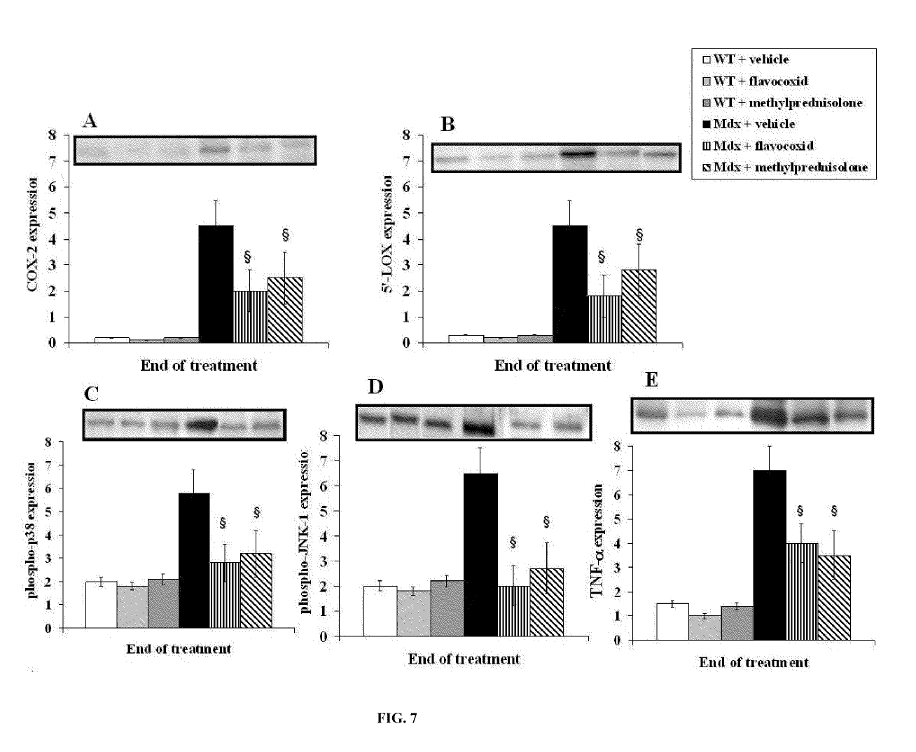

As shown in FIG. 7, a very low level of expression of COX-2,5-LOX, TNF-α, phospho-JNK-1, and phospho-p38 was detected in WT animals treated either with vehicle, flavocoxid or methylprednisolone. By contrast, vehicle-treated mdx mice showed an increased expression of these inflammatory molecules when compared to WT animals. Treatments with either flavocoxid or methylprednisolone was able to significantly reduce (p<0.01) COX-2,5-LOX, phospho-JNK-1, phospho-p38 and TNF-α expression in mdx mice.

### Example 2

### Treatment of Wild-Type and MDX Mice with Genistein

### Materials and Methods

Animals

Mdx animals were purchased from Charles River Italy (Calco, Milan, Italy). Animals were 4 weeks old on arrival and were acclimatized for 5 days before the study. At the beginning of the experiment the mice, which were at that point 5 weeks old, weighted about 20-22 grams. The mice were treated daily for 5 weeks with either the phytoestrogen genistein (Sigma, Mo., USA) at a dose of 2 mg/kg/ip or its vehicle (1:3 DMSO: 0.9% NaCl solution).

Statistical Analysis

Results were expressed as mean±SD. Statistical comparison between treated and control groups was performed by the 2-tailed Student's t-test on paired samples with the use of InPlotPrism software version 3.0 (GraphPad Software, San Diego, Calif., USA). P values<0.05 were considered significant.

### Results

Strength Examinations

Mice were weighed and examined for forelimb strength at baseline and at the end of the experiment. Strength testing consisted of five separate measurements using a grip meter attached to a force transducer that measures the peak force generated (Stoelting Co., Wooddale Ill., USA). Specifically, the mouse grabbed the trapeze bar as it is pulled backward and the peak pull force in grams is recorded on a display. The three highest measurements for each animal were averaged to give the strength score.

The degree of fatigue was calculated by comparing the first two pulls to the last two pulls. The decrease between pulls 1+2 and pulls 4+5 provided the measure of fatigue.

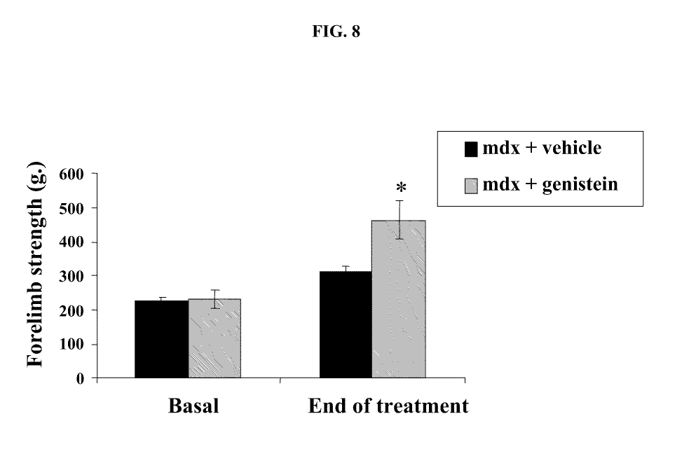

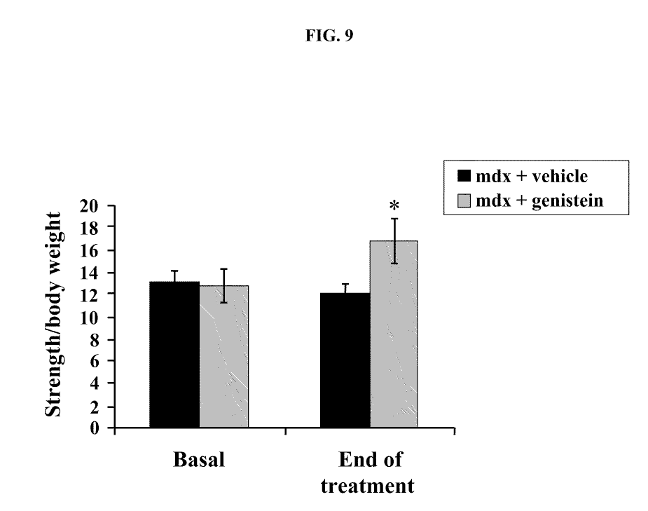

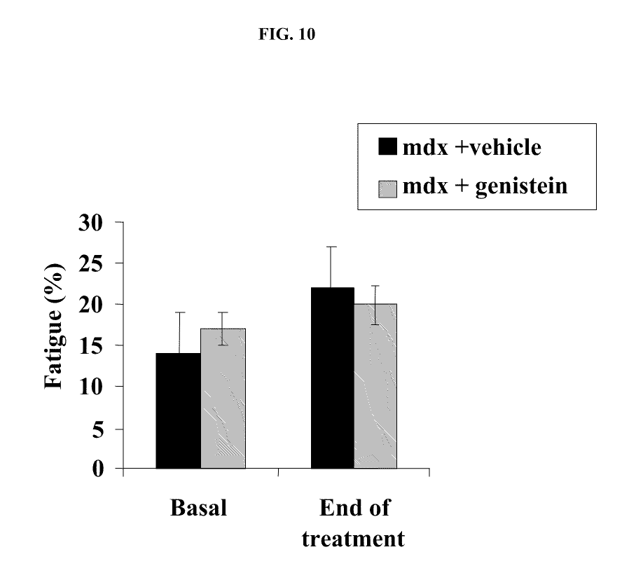

Body weight was not significantly different between the genistein and vehicle treated mice. As shown in FIG. 8, genistein treatment significantly augmented forelimb strength as compared to vehicle treatment (p<0.01). As shown in FIG. 9, genistein treated mice had a higher strength normalized to weight (p<0.01) as compared to vehicle-treated mice. As shown in FIG. 10, genistein treated mice had a slightly lower level of fatigue as compared to vehicle-treated mice.

Serum Creatine Kinase (CK) Evaluation

Blood samples were centrifuged at 6000 g and the serum was stored at −80° C. until the day of analysis. Serum CK was evaluated at 37° C. using a commercially available kit (Randox Laboratories LTD, Antrim, UK). The results are expressed as U/L.

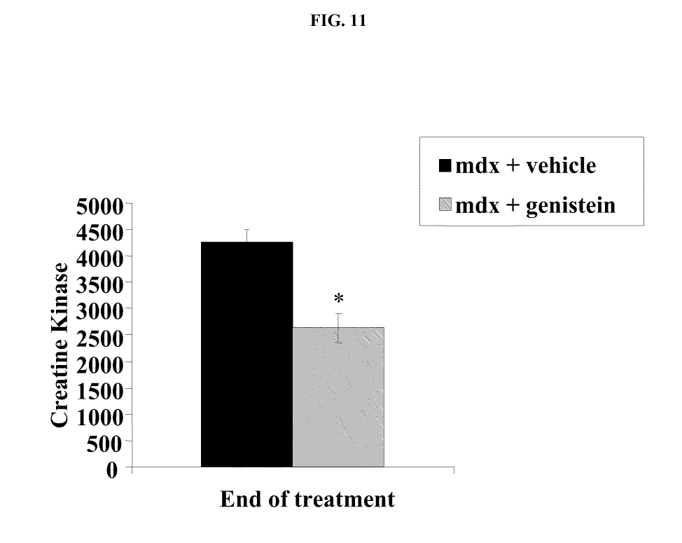

As shown in FIG. 11, treatment with genistein resulted in a statistically significant reduction of the CK levels as compared to vehicle treatment (p<0.01).

Histological Studies

Transverse cryostat sections 10 μm thick were obtained from the midpoint of the biceps and TA muscle body. The whole muscle cross sections (corresponding to a mean area of 2.10 mm² in biceps and 2.25 mm² in TA) were stained with hematoxylin-eosin and were examined by a blinded observer using the AxioVision 2.05 image analysis system equipped with an Axiocam camera scanner (Zeiss, Munchen, Germany). The following four areas with patchy distribution were recognized: 1) normal fibers identified by the presence of peripheral nuclei; 2) necrotic fibers identified by pale cytoplasm and phagocytosis; 3) regenerating fibers identified by small size, central nuclei with/without basophilic cytoplasm; 4) centrally nucleated fibers identified by normal size but with central nuclei. The area occupied by normal fibers, necrotic fibers, regenerating fibers, or centrally nucleated fibers was expressed as a percentage of the area of the whole muscle cross section.

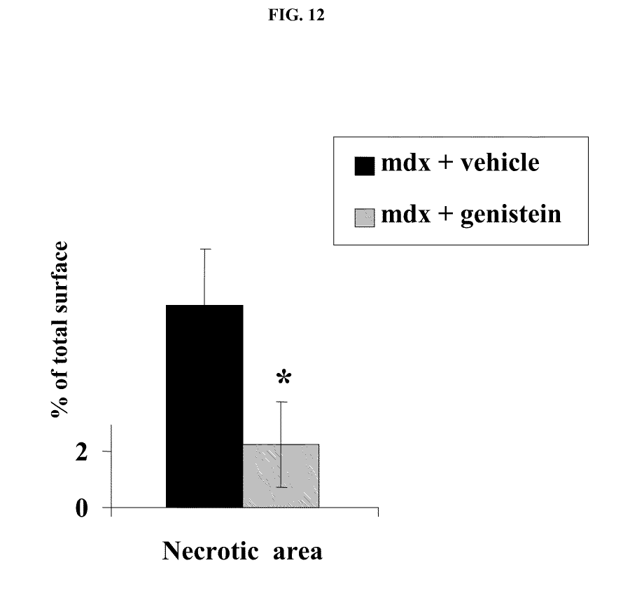

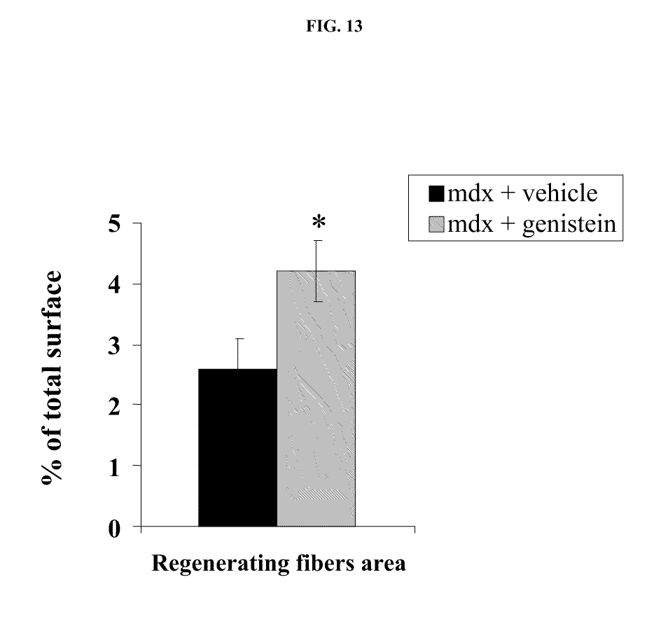

Genistein treatment was not found to modify the percentage of normal muscular area or centrally nucleated fibers in the biceps as compared to vehicle treatment. As shown in FIG. 12, genistein treatment decreased necrosis in the biceps as compared to vehicle treatment (p<0.05). As shown in FIG. 13, genistein treatment increased the percentage of regenerating fibers in the biceps as compared to vehicle treatment (p<0.05).

Immunohistochemistry

Immunohistochemical detection of specific antigens was performed on 7 μm-thick transverse cryostat sections from biceps muscles. A rabbit polyclonal antibody against:myogenin (1:60; #sc-576, Santa Cruz Biotechnology, Inc., Santa Cruz, Calif., USA) and a mouse monoclonal antibody against developmental myosin heavy chain (dMHC) (1:20; #NCL-MHCd, Novocastra Laboratories Ltd., Newcastle on Tyne, UK) were used to study regeneration. Myogenin is expressed by differentiating myoblasts and is considered an “early” marker of regeneration. dMHC was used as a marker of “later” regeneration. Visualization of antibody staining was achieved using a peroxidase detection system (ABComplex/HRP kit, Dako, Milan, Italy) and the 3,3-diaminobenzidine tetrahydrochloride as chromogen. In the case of dMHC, the AEC substrate chromogen (Zymed Laboratories, San Francisco, Calif., USA) was used for color development.

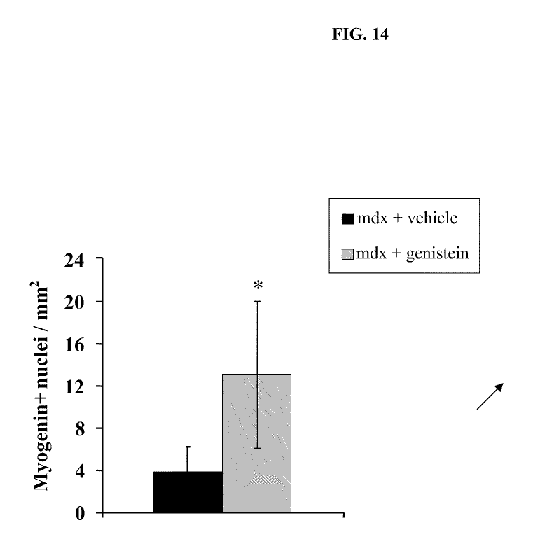

Genistein treatment was not found to modify either the number of myogenin positive satellite cells (SC) and of dMHC positive fibers in mdx animals compared to vehicle treatment. As shown in FIG. 14, genistein treatment significantly increased myogenin expression in the nuclei of muscles as compared to vehicle treatment (p<0.05).

NF-κB Activation

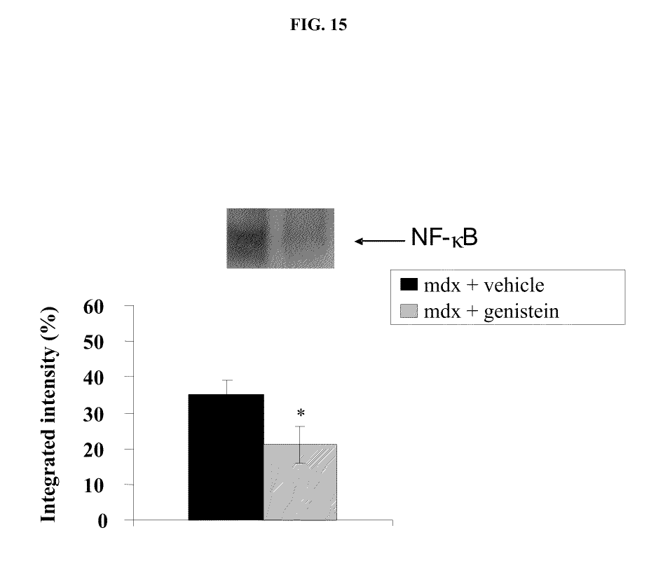

Isolation of nuclear proteins in approximately 50 mg of frozen skeletal muscle tissue was performed according to recently detailed method (See ref 15 below). Twenty micrograms of nuclear extract were incubated for 30 min at room temperature with 50 fmol of biotin-end-labeled 45-mer double-strand NF-κB oligonucleotide from the HIV long terminal repeat, 5-TTGTTACAAGGGACTTTCCGCTGGGGACTTTCCAGGA GGCGTGGG-3 containing 2(underlined) NF-κB binding sites. Both complimentary oligos were end-labeled separately and then annealed prior to use. Binding reaction mixtures were prepared in a final volume of 20 μL HEPES buffer containing 1 mg double-strand poly dI/dC, 10% glycerol, 100 mM MgCl₂ and 1% Nonidet P-40, performed with the LightShif™ Chemiluminescent EMSA Kit (Pierce, Milan, Italy), according to the manufacturer s instructions. Bound complexes were separated on 7.5% nondenaturating polyacrylamide gels, blotted onto nylon membrane and visualized on Kodak X-ray film (Kodak, Milan, Italy) by autoradiography. The results were expressed as relative integrated intensity compared with normal controls, considering exposure time, background levels and known protein concentration of an Epstein Barr virus nuclear antigen-1 extract, which was used as EMSA control. As shown in FIG. 15, genistein treatment significantly decreased NF-κB activation as compared to vehicle treatment (p<0.05).

Electrophysiological Studies

Extensor digitorum longus muscles (EDL) were vertically mounted in a temperature controlled (30°) chamber containing Krebs Ringer bicarbonate buffer (Sigma k4002) and continuously gassed with a mixture of 95% O2 and 5% CO2. One end of the muscle was linked to a fixed clamp while the other end was connected to the lever-arm of an Aurora Scientific Instruments 300B actuator/transducer system using a nylon wire. The isolated muscle was electrically stimulated with 200 mA controlled current pulses (which resulted in a pulse voltage of around 10V) by means of two platinum electrodes located 2 mm from each side of the muscle. For each experiment, the initial muscle length was adjusted to the length (L0) which produced the highest twitch force. The optimal fiber length (Lf) was determined by multiplying the value of L0 for the fiber length by the muscle length ratio (0.44 for the EDL and 0.71 for soleus). The muscle cross-sectional area (CSA) was determined for the EDL and soleus by dividing the muscle mass (m) by the product of the Lf and the density of mammalian skeletal muscle (1.06 mg/mm³).

In order to measure force generation, the muscle was initially stimulated with two 0.5 ms single pulses (with a rest time of 150 s) to measure twitch force, twitch force/cross sectional area (CSA), and contraction time (the time that the isolated muscle needs to reach the peak force after delivery of a single electrical pulse).

In order to measure high-stress eccentric contractions, the muscle was stimulated with five tetanic stimulations (train of 0.1 ms pulses delivered at 180 Hz, for 0.6 s) to evaluate the maximum isometric force (and maximum force/cross sectional area (CSA)) and the decline in maximal isometric force. The latter was measured by dividing the force value at the last stimulation (F5) by the force value at the first stimulation (F1), and used as a index of myofiber injury caused by high-stress contractions.

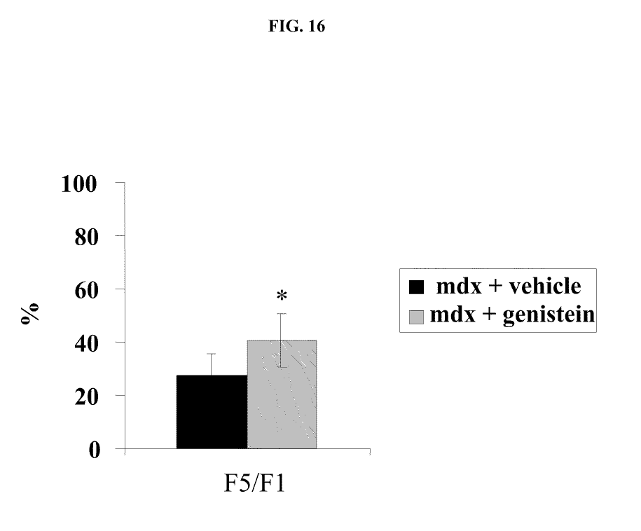

Genistein treatment was not found to modify either twitch force, twice force/CSA, contraction time, or maximum force/CSA as compared to vehicle treatment. As shown in FIG. 16, genistein treatment reduced the decline of maximal isometric force in muscles as compared to vehicle treatment (p<0.05).

### REFERENCES

The entire disclosure of publications mentioned herein are incorporated by reference.
(1) Dubowitz V: Muscle Disorders in Childhood, 2nd edition. W.B. Saunders, 1995.
(2) Emery E H: The Muscular Dystrophies. The Lancet, 2002. 359: p. 687-695.
(3) G. Danialou, A. S. Comtois, R. Dudley, G. Karpati, G. Vincent, C. Des Rosiers and B. J. Petrof, Dystrophin-deficient cardiomyocytes are abnormally vulnerable to mechanical stress-induced contractile failure and injury, FASEB J. 15 (2001) (9), pp. 1655-165.
(4) B. J. Petrof, J. B. Shrager, H. H. Stedman, A. M. Kelly and H. L. Sweeney, Dystrophin protects the sarcolemma from stresses developed during muscle contraction, Proc. Natl. Acad. Sci. U.S.A. 90 (1993) (8), pp. 3710-3714.
(5) M. E. Murphy and J. P. Kehrer, Oxidative stress and muscular dystrophy, Chem. Biol. Interact. 69 (1989) (2-3), pp. 101-178.
(6) R. J. Ragusa, C. K. Chow and J. D. Porter, Oxidative stress as a potential pathogenic mechanism in an animal model of Duchenne muscular dystrophy, Neuromuscul. Disord. 7 (1997) (6-7), pp. 379-386.
(7) L. Austin, M. de Niese, A. Mc Gregor, H. Arthur, A. Gurusinghe and M. K. Gould, Potential oxyradical damage and energy status in individual muscle fibres from degenerating muscle diseases, Neuromuscul. Disord. 2 (1992) (1), pp. 27-33.
(8) Karin M: The beginning of the end: IkappaB kinase (IKK) and NF-kappaB activation. J Biol Chem 1999, 274:27339-27342.
(9) Karin M, Delhase M: The I kappa B kinase (IKK) and NF-kappa B: key elements of proinflammatory signaling. Semin Immunol 2000, 12:85-98.
(10) Baldwin A S Jr. Series introduction: the transcription factor NF-kappaB and human disease. J Clin Invest. 2001 January; 107(1):3-6.
(11) Kumar, Y. Takada, A. M. Boriek and B. B. Aggarwal, Nuclear factor-kappaB: its role in health and disease, J. Mol. Med. 82 (2004) (7), pp. 434-448.
(12) M. Karin, The beginning of the end: IkappaB kinase (IKK) and NF-kappaB activation, J. Biol. Chem. 274 (1999) (6362), pp. 27339-27342.
(13) U. Senftleben and M. Karin, The IKK/NF-kappa B pathway, Crit. Care Med. 30 (2002) (1 Suppl.), pp. S18-S26.
(14) M. Karin and M. Delhase, The I kappa B kinase (IKK) and NF-kappa B: key elements of proinflammatory signalling, Semin. Immunol. 12 (2000) (1), pp. 85-98.
(15) M. C. Monici, M. Aguennouz, A. Mazzeo, C. Messina and G. Vita, Activation of nuclear factor-kappaB in inflammatory myopathies and Duchenne muscular dystrophy, Neurology 60 (2003) (6), pp. 993-997.
(16) Kumar, Y. Takada, A. M. Boriek and B. B. Aggarwal, Nuclear factor-kappaB: its role in health and disease, J. Mol. Med. 82 (2004) (7), pp. 434-448.
(17) Messina S, Altavilla D, Aguennouz M, Seminara P, Minutoli L, Monici M C, Bitto A, Mazzeo A, Marini H, Squadrito F, Vita G. Lipid peroxidation inhibition blunts nuclear factor-kappaB activation, reduces skeletal muscle degeneration, and enhances muscle function in mdx mice. Am J Pathol. 2006 March; 168(3):918-26.
(18) Messina S, Bitto A, Aguennouz M, Minutoli L, Monici M C, Altavilla D, Squadrito F, Vita G. Nuclear factor kappa-B blockade reduces skeletal muscle degeneration and enhances muscle function in Mdx mice. Exp Neurol. 2006 March; 198(1):234-41.
(19) E. Porreca, M. D. Guglielmi, A. Uncini, P. Di Gregorio, A. Angelici, C. Di Febbo, S. D. Pierdomenico, G. Baccante and F. Cuccurullo, Haemostatic abnormalities, cardiac involvement and serum tumor necrosis factor levels in X-linked dystrophic patients, Thromb. Haemost. 81 (1999) (4), pp. 543-546.
(20) N. Watanabe, Clinical significance of measurement of circulating tumor necrosis factor alpha, Rinsho Byori 49 (2001) (9), pp. 829-833.
(21) D. Thaloor, K. J. Miller, J. Gephart, P. O. Mitchell and G. K. Pavlath, Systemic administration of the NF-κB inhibitor curcumin stimulates muscle regeneration after traumatic injury, Am. J. Physiol. 277 (1999) (2 Pt. 1), pp. C320-C329.
(22) Monici M C, Aguennouz M, Mazzeo A, Messina C, Vita G: Activation of nuclear factor-kappaB in inflammatory myopathies and Duchenne muscular dystrophy. Neurology 2003, 60:993-997.
(23) Kumar A, Boriek A M: Mechanical stress activates the nuclear factor-kappaB pathway in skeletal muscle fibers: a possible role in Duchenne muscular dystrophy. FASEB J 2003, 17:386-396.
(24) Gong G, Waris G, Tanveer R, Siddiqui A: Human hepatitis C virus NS5A protein alters intracellular calcium levels, induces oxidative stress, and activates STAT-3 and NF-kappa B. Proc Natl Acad Sci USA 2001, 98:9599-9604.
(25) Muller J M, Rupec R A, Baeuerle P A: Study of gene regulation by NF-kappa B and AP-1 in response to reactive oxygen intermediates. Methods 1997, 11:301-312.
(26) Lee J and Burckart G J. Nuclear factor kappa B: important transcription factor and therapeutic target. J Clin Pharmacol 1998; 38:981-993.
(27) Tidball J G. Inflammatory processes in muscle injury and repair. Am J Physiol—Regulatory Integrative and Comp Physiol 2005. 288(2):R345-53.
(28) Nakashima J, Tachibana M, Ueno M, Miyajima A, Baba S, Murai M: Association between tumor necrosis factor in serum and cachexia in patients with prostate cancer. Clin Cancer Res 1998, 4:1743-1748.
(29) Zhao S P, Zeng L H: Elevated plasma levels of tumor necrosis factor in chronic heart failure with cachexia. Int J Cardiol 1997, 58:257-261.
(30) Grounds M D, Torrisi J: Anti-TNFalpha (Remicade) therapy protects dystrophicskeletal muscle from necrosis. FASEB J 2004, 18:676-682.
(31) Kyriakis J M, Avruch J. Mammalian mitogen-activated protein kinase signal transduction pathways activated by stress and inflammation. Physiol Rev. 2001 April; 81(2):807-69.
(32) M. Guha and N. Mackman, LPS induction of gene expression in human monocytes, Cell Signal 13 (2001), pp. 85-94.
(33) N. R. Bhat, P. Zhang, J. C. Lee and E. L. Hogan, Extracellular signal regulated kinase and p38 subgroups of mitogen-activated protein kinases regulate inducible nitric oxide synthase and tumor necrosis factor-alpha gene expression in endotoxin-stimulated primary glial cultures, J Neurosci 18 (1998), pp. 1633-1641.
(34) Kolodziejczyk S M, Walsh G S, Balazsi K et al. Activation of JNK1 contributes to dystrophic muscle pathogenesis. Curr. Biol. 2001; 11: 1278-82.
(35) Hnia K, Hugon G, Rivier F et al. Modulation of p38 mitogen-activated protein kinase cascade and metalloproteinase activity in diaphragm muscle in response to free radical scavenger administration in dystrophin-deficient Mdx mice. Am. J. Pathol. 2007; 170: 633-43.
(36) Messina S, Altavilla D, Aguennouz M, Seminara P, Minutoli L, Monici M C, Bitto A, Mazzeo A, Marini H, Squadrito F, Vita G. Lipid peroxidation inhibition blunts nuclear factor-kappaB activation, reduces skeletal muscle degeneration, and enhances muscle function in mdx mice. Am J Pathol. 2006 March; 168(3):918-26.
(37) Messina S, Bitto A, Aguennouz M, Minutoli L, Monici M C, Altavilla D, Squadrito F, Vita G. Nuclear factor kappa-B blockade reduces skeletal muscle degeneration and enhances muscle function in Mdx mice. Exp Neurol. 2006 March; 198(1):234-41.
(38) Dorchies O M, Wagner S, Vuadens O, Waldhauser K, Buetler T M, Kucera P, Ruegg U T. Green tea extract and its major polyphenol (−)-epigallocatechin gallate improve muscle function in a mouse model for Duchenne muscular dystrophy. Am J Physiol Cell Physiol. 2006 February; 290(2):C616-25.
(39) Nakae Y, Hirasaka K, Goto J, Nikawa T, Shono M, Yoshida M, Stoward P J. Subcutaneous injection, from birth, of epigallocatechin-3-gallate, a component of green tea, limits the onset of muscular dystrophy in mdx mice: a quantitative histological, immunohistochemical and electrophysiological study. Histochem Cell Biol. 2008 April; 129(4):489-501.
(40) The Combined Chemical Dictionary, Chapman & Hall/CRC, Version 5:1 June 2001.
(41) Heo et al. (2001) Mutat. Res. 488(2):135-150.
(42) Kubo et al. (2000) Bioorg. Med. Chem. 8(7):1749-1755.
(43) Imamura et al. (2000) J. Biochem. 127(4):653-658.
(44) Afolayan and Meyer (1997) Ethnopharmacol. 57(3):177-181.
(45) Meyer et al. (1997) J. Ethnopharmacol. 56(2):165-169),
(46) So et al. (1997) Cancer Lett. 112(2):127-133.

What is claimed is:

1) A method of treating muscular dystrophies, comprising:
a) providing a pharmaceutical composition comprising a therapeutically effective amount of flavonoid; and
b) administering the composition to a human patient;
wherein the flavonoid comprises an isoflav-4-one nucleus, and
wherein at least one of the carbons of the nucleus located at positions 8, 7, 6, 5, 2, 2′, 3′, 4′, 5′, or 6′ is modified by an alcohol group.

2) A method of treating muscular dystrophies, comprising:
a) providing a pharmaceutical composition comprising a therapeutically effective amount of flavonoid; and
b) administering the composition to a human patient;
wherein the flavonoid comprises a flavan-3-ol nucleus, and
wherein at least one of the carbons of the nucleus located at positions 8, 7, 6, 5, 4, 2′, 3′, 4′, 5′, or 6′ is modified by an alcohol group.

3) A method of treating Duchenne muscular dystrophy, comprising:
a) providing a pharmaceutical composition comprising a therapeutically effective amount of flavonoid; and
b) administering the composition to a human patient;
wherein the flavonoid comprises a Free-B-Ring flavone nucleus,
wherein at least one of the carbons of the nucleus located at positions 8, 7, 6, 5 or 3 is modified by an alcohol group, and
wherein at least one of the carbons of the nucleus located at positions 8, 7, 6, 5 or 3 is modified by a glycoside group.

4) The method of claim 1 wherein the muscular dystrophy is Duchenne's muscular dystrophy.

5) The method of claim 2 wherein the muscular dystrophy is Duchenne's muscular dystrophy.

6) The method of claim 3 wherein the muscular dystrophy is Duchenne's muscular dystrophy.

7) The method of claim 1 wherein the step of administering the composition comprises administering said flavonoid in an amount in the range of about 250 to about 1000 mg per day.

8) The method of claim 2 wherein the step of administering the composition comprises administering said flavonoid in an amount in the range of about 250 to about 1000 mg per day.

9) The method of claim 3 wherein the step of administering the composition comprises administering said flavonoid in an amount in the range of about 250 to about 1000 mg per day.

10) The method of claim 1, wherein the carbons at positions 5, 7, and 4′ are modified by alcohol groups.

11) The method of claim 10, wherein the carbons located at positions 8, 7, 6, 5, 2, 2′, 3′, 4′, 5′, and 6′ are not modified by any functional groups that are not alcohol groups.

12) The method of claim 1, wherein the flavonoid comprises genistein.

13) The method of claim 2, wherein the number of carbons of the nucleus modified by alcohol groups is between two and six.

14) The method of claim 13, wherein the carbons at positions 5, 7, 3′, and 4′ are modified by alcohol groups.

15) The method of claim 2, wherein the flavonoid comprises at least one of catechin or epicatechin.

16) The method of claim 3, wherein the number of carbons of the nucleus modified by alcohol groups is between one and three.

17) The method of claim 16, wherein the carbons at positions 5 and 6 are modified by alcohol groups.

18) The method of claim 3, wherein the glycoside group comprises a pyranose.

19) The method of claim 3, wherein the flavonoid comprises baicalin.

20) The method of claim 19, wherein the pharmaceutical formulation further comprises a therapeutically effective amount of second flavonoid selected from catechin or epicatechin.
